(19) United States
(12) Patent Application Publication
(10) Pub. No.: US 2025/0005388 A1
(43) Pub. Date: Jan. 2, 2025

(54) PREDICTIVE MODEL-GENERATING SYSTEM, PREDICTIVE MODEL-GENERATING METHOD, AND PREDICTION METHOD

(71) Applicant: NIPPON TELEGRAPH AND TELEPHONE CORPORATION, Tokyo (JP)

(72) Inventors: Sosuke IMAMURA, Musashino-shi (JP); Kazuhiro TAKAYA, Musashino-shi (JP)

(73) Assignee: NIPPON TELEGRAPH AND TELEPHONE CORPORATION, Tokyo (JP)

(21) Appl. No.: 18/709,523

(22) PCT Filed: Nov. 19, 2021
(86) PCT No.: PCT/JP2021/042617

§ 371 Date: May 13, 2024

(51) Int. Cl. G06N 5/022

(52) U.S. Cl. CPC G06N 5/022

(57) ABSTRACT

A technology for enabling efficient growing or breed improvement is provided. A predictive model generating system includes a genome-editing apparatus, a growing system, and a processing apparatus. The processing apparatus acquires genome-editing data from the genome-editing apparatus, acquires growth environment data and growth situation data in each growing apparatus from the growing system, generate teaching data from these data, causes a machine-learning model for predicting a growth situation or mutagenesis situation of a living body from the genome mutation and the growth environment or predicting the genome mutation from the growth environment and the growth situation of the living body, to perform learning with the teacher data, and obtains the machine-learning model that has completed the learning as a predictive model.

### TECHNICAL FIELD

The present invention relates to the creation of a predictive model.

### BACKGROUND ART

Research is being conducted to find the optimal growth environment for new breeds created by genome editing by monitoring the growth situation in plant factories. Further, research is being conducted to find the relationship between cultivation conditions and the growth situation and estimate growth results.

For example, a cultivation evaluation system for a genetically modified plant described in PTL 1 includes an isolated cultivation field in which a plant is cultivated in a state in which the plant is isolated from the outside world, a network that enables exchange of information between an administrator of this isolated cultivation field and a genetically modified plant researcher, and a server system that collects and distributes information via this network. The server system has an isolated cultivation field management function of assigning an available isolated cultivation field or a part thereof to the researcher, and a plant cultivation process management function of cultivating the genetically modified plant through a cultivation process according to an instruction of the researcher in the isolated cultivation field or the part that is being used. The use of this cultivation evaluation system makes it possible to easily check a current status of the genetically modified plant entrusted by the researcher who is a client, and to efficiently cultivate the genetically modified plant in this isolated cultivation field.

A method of predicting a yield of crops described in PTL 2 includes a yield prediction step of inputting a value of an environment factor to a relational expression of the environment factor in cultivation conditions of a crop and a light utilization efficiency of the crop to calculate a value of the light utilization efficiency of the crop corresponding to the value of the environment factor, and predicting a yield of the crop from the calculated value of the light use efficiency of the crop. According to this method, in cultivation of crops, such as cultivation of crops in a crop cultivation apparatus or a plant factory, optimization of the cultivation conditions and the prediction of the yield can be performed easily and quickly.

PTL 3 describes a characteristics estimation model generation apparatus. This characteristics estimation model generation apparatus generates a model that estimates a characteristics variable from a state variable representing a state of an analysis target and the characteristics variable representing characteristics of the analysis target. This characteristic estimation model generation apparatus includes a data output unit to which the state variable of the analysis target and the characteristics variable of the analysis target are input and that outputs analysis data, a regression analysis unit that performs regression analysis having a regularization term with the characteristics variable in the analysis data as an objective variable and the state variable as an explanatory variable, to generate a regression model representing a relationship between the objective variable and the explanatory variable, a characteristic estimation model generation unit that performs cross-validation up to a preset number of verifications and generates a model having an optimal regularization term among the regression models as a characteristic estimation model, and an analysis data updating unit that generates, as update data, data obtained by excluding data corresponding to the explanatory variables selected in the characteristic estimation model from the analysis data, and outputs the data to the data output unit as the analysis data at the time of the next characteristic estimation model generation. This characteristic estimation model generation apparatus repeats the updating of the analysis data and the generation of the characteristic estimation model using the updated data up to a preset number of repetitions. According to this characteristic estimation model generation apparatus, it is possible to comprehensively extract state variables related to the characteristics variable of the analysis target.

PTL 3 also describes a characteristic estimation apparatus that estimates the characteristic of the analysis target. This characteristic estimation apparatus includes a characteristic estimation unit that outputs characteristic estimation information, which is a result of estimating the characteristics of the analysis target, by gene expression information of a creature input to the characteristic estimation model generated by the characteristic estimation model generation apparatus. This characteristic estimation apparatus can further include a state diagnosis unit that diagnoses a state of the creature using the characteristic estimation information.

A cohort phenotyping system for plant factory with artificial lighting described in PTL 4 includes a calculation and control unit, a data set input unit, a data storage, a machine-learning unit, an image-processing unit, a statistical data analysis unit, an association and causal relation derivation calculation unit, and an integrated control unit having a verification unit. On the basis of image information, environment factor information, genetic characteristic information, and artificial operation information input from the data set input unit and stored in the data storage, the integrated control unit continuously measures plant characteristic information in a growth process non-destructively in a germination period to calculate a two-dimensional distribution of plant characteristics, continuously measures a physiological performance reaction of seeds sown in an environmentally controlled closed space in the germination period, and continuously measures a two-dimensional distribution of nutrient solution temperature, air temperature, saturation, nutrient solution rate, pH, and electrical conductivity. This makes it possible to reduce labor and work time for automating production in a plant factory and automating seed selection and growing work in seedling production, thereby improving a germination rate and producing uniform seedlings.

NPL 1 describes results of research aimed at obtaining basic knowledge necessary to develop advanced cultivation technology for curbing bolting and late bolting breeds, by identifying genes related to bolting control in lettuce and clarifying their functions. NPL 1 describes that a homologous gene (LsFT gene) in lettuce of the FLOWERING LOCUS T (FT) gene, which is a flowering control gene isolated in many plants has been isolated, that a translation region of complementary DeoxyriboNucleic Acid (cDNA) in the LsFT gene is 74-84% homologous at an amino acid level with FT-like genes of other plants such as Arabidopsis thaliana, that bolting is promoted in Arabidopsis thaliana in which the A LsFT gene is overexpressed, and that it has been found that an expression amount of LsFT gene fluctuates during one day and increases with the differentiation and development of flower buds.

### CITATION LIST

### Patent Literature

[PTL 1] Japanese Patent Application Publication No. 2004-121093
[PTL 2] Japanese Patent Application Publication No. 2021-45063
[PTL 3] Japanese Patent Application Publication No. 2017-51118
[PTL 4] WO2020/170939

### Non Patent Literature

[NPL 1] https://www.naro.go.jp/project/results/laboratory/vegetea/2011/113a4_10_04.html.

### SUMMARY OF INVENTION

An object of the present invention is to provide a technology for enabling efficient growing and breed improvement.

According to a first aspect of the present invention, there is provided a predictive model-generating system including: a growing system including one or more growing apparatuses, each of the one or more growing apparatuses including an environment chamber configured to grow one or more of a plurality of living bodies respectively derived from a plurality of mutants, an environment control apparatus configured to control an environment in the environment chamber, and a growth situation-monitoring apparatus configured to monitor a growth situation of the living body in the environment chamber, and the one or more growing apparatuses being for growing each of the plurality of living bodies under different environments; and a processing apparatus configured to acquire growth environment data and growth situation data in each of the one or more growing apparatuses from the growing system, generate teacher data from the genome mutation data of the plurality of mutants, the growth environment data, and the growth situation data, cause a machine-learning model for predicting the growth situation or mutagenesis situation of the living body from the genome mutation and the growth environment or predicting the genome mutation from the growth environment and the growth situation of the living body, to perform learning with the teacher data, and obtain the machine-learning model that has completed the learning as a predictive model.

According to a second aspect of the present invention, there is provided the predictive model-generating system according to the first aspect, further including: a genome-editing apparatus configured to generate the plurality of mutants through genome editing.

According to a third aspect of the present invention, there is a provided a predictive model-generating system according to the first aspect, further including: a genome mutagenesis-processing apparatus configured to generate a plurality of processing bodies containing the plurality of mutants through genome mutagenesis processing; and a genome mutation situation analysis apparatus configured to analyze the genome mutation situation of each of the plurality of processing bodies.

According to a fourth aspect of the present invention, there is provided a predictive model-generating system including: a growing system including one or more growing apparatuses, each of the one or more growing apparatuses including an environment chamber configured to grow living bodies, an environment control apparatus configured to control an environment in the environment chamber, and a growth situation-monitoring apparatus configured to monitor a growing condition of the living body in the environment chamber, and the one or more growing systems includes a growing system configured to grow the living body under different environments; a genome mutation situation analysis apparatus configured to analyze a genome mutation situation for the living bodies grown in the one or more growing apparatuses; and a processing apparatus configured to acquire genome mutation situation data from the genome mutation situation analysis apparatus, acquire growth environment data and growth situation data in each of the one or more growing apparatuses from the growing system, generate teacher data from the genome mutation data, the growth environment data, and the growth situation data, cause a machine-learning model for predicting the growth situation or mutagenesis situation of the living body from the genome mutation and the growth environment or predicting the genome mutation from the growth environment and the growth situation of the living body, to perform learning with the teacher data, and obtain the machine-learning model that has completed the learning as a predictive model.

According to the fifth aspect of the present invention, there is a provided a method of generating a predictive model including: growing a plurality of living bodies respectively derived from a plurality of mutants under different environments; and acquiring genome mutation data for the plurality of mutants, acquiring growth environment data and growth situation data of the plurality of living bodies, generating teacher data from the genome mutation data, the growth environment data, and the growth situation data, causing a machine-learning model for predicting the growth situation or mutagenesis situation of the living body from the genome mutation and the growth environment, or predicting the genome mutation from the growth environment and the growth situation of the living body, to perform learning with the teacher data, and obtaining the machine-learning model that has completed the learning as a predictive model.

According to the sixth aspect of the present invention, there is a provided a method of generating a predictive model including: growing living bodies in different environments; analyzing a genome mutation situation for the grown living body; and acquiring genome mutation situation data of the grown living body, acquiring growth environment data and growth situation data of the living body, generating teacher data from the genome mutation situation data, the growth environment data, and the growth situation data, causing a machine-learning model for predicting the growth situation or mutagenesis situation of the living body from the genome mutation and the growth environment or predicting the genome mutation from the growth environment and the growth situation of the living body, to perform learning with the teacher data, and obtaining the machine-learning model that has completed the learning as a predictive model.

According to a seventh aspect of the present invention, there is provided a prediction method including: predicting the growth situation or mutagenesis situation of the living body or genome mutation using the predictive model generated by the generation method according to the fifth or sixth aspect.

According to this invention, a technique enabling efficient growing and breed improvement is provided.

### BRIEF DESCRIPTION OF DRAWINGS

FIG. 1 is a block diagram of a predictive model-generating system according to a first embodiment of the present invention.

FIG. 2 is a flowchart of a predictive model-generating method according to the first embodiment of the present invention.

FIG. 3 is a block diagram of a predictive model generating system according to a second embodiment of the present invention.

FIG. 4 is a flowchart of the predictive model-generating method according to the second embodiment of the present invention.

FIG. 5 is a block diagram of a predictive model-generating system according to a third embodiment of the present invention.

FIG. 6 is a flowchart of a predictive model-generating method according to the third embodiment of the present invention.

### DESCRIPTION OF EMBODIMENTS

Hereinafter, embodiments of the present invention are described. Embodiments which are described below are more specific embodiments of any one of the above aspects. Matters which are described below can be incorporated into each of the above aspects alone or in combination.

### <1> First Embodiment

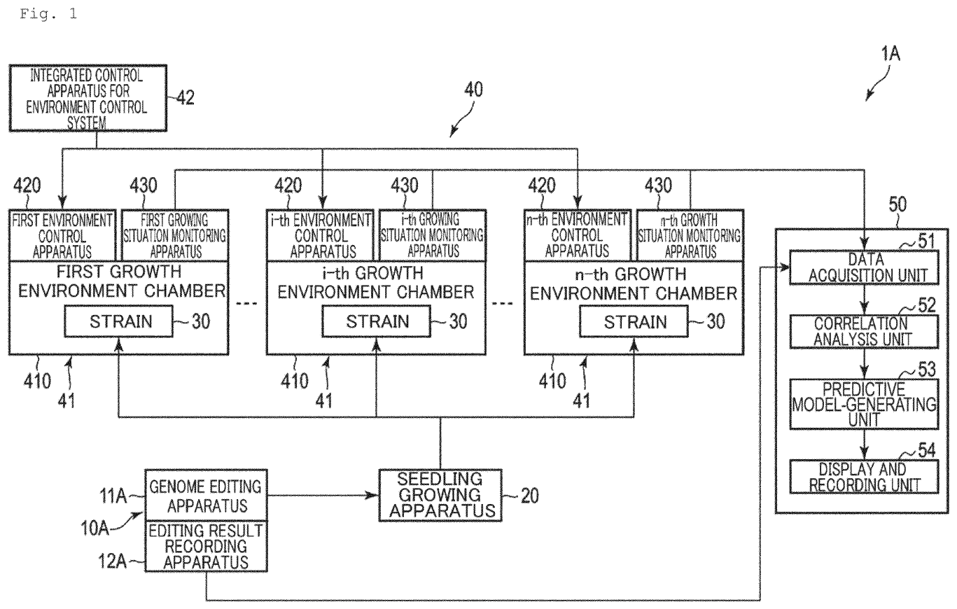

FIG. 1 is a block diagram of a predictive model-generating system according to a first embodiment of the present invention. A predictive model-generating system 1A illustrated in FIG. 1 is a system that creates a predictive model for predicting a growth situation of plants or a mutagenesis situation. The predictive model-generating system 1A includes a genome-editing unit 10A, a seedling-growing apparatus 20, a growing system 40, and a processing apparatus 50.

The genome-editing unit 10A includes a genome-editing apparatus 12A and an editing result-recording apparatus 12A.

The genome-editing apparatus 11A generates a plurality of mutants with different genetic information through genome editing of the living body (here, a plant).

The genome-editing apparatus 11A includes a mutagenesis apparatus that introduces a site-specific nuclease or the like into a cell, uses this the site-specific nuclease to cleave genomic DNA at an arbitrary position, and causes a repair error such as deletion, substitution, and insertion of bases at a cleaving position at the time of repair, or inserts a specific sequence as a fragment at the cleaving position. For this cleavage, for example, Clustered Regularly Interspaced Short Palindromic Repeats (CRISPR)/Crispr Associated protein 9 (Cas9), Zinc-Finger Nuclease (ZFN), or Transcription Activator-Like Effector Nuclease (TALEN) can be used.

Introduction of the site-specific nuclease or the like into plant cells is performed on, for example, a plant body, a tissue such as a shoot apical meristem, or a callus.

For introduction of the site-specific nuclease or the like into the cells, for example, a physical scheme such as a particle gun method and an electroporation method can be used.

In the particle gun method, composite particles containing carrier particles such as gold particles and a site-specific nuclease or the like carried thereon are injected toward the cells using, for example, high-pressure gas, and accordingly, site-specific nuclease or the like is introduced into the cell. In this case, the mutagenesis apparatus may include a holder that holds composite particles, and an airflow generation apparatus that supplies a high-speed airflow toward the holder to move the composite particles from the holder to cells at high speed. Here, in the case of CRISPR/Cas9, for example, the composite particles are composite particles in which a complex of Cas9 and single guide Ribonucleic Acid (sgRNA) is carried on the carrier particles. The airflow generation apparatus may include at least one of a pump and a gas cylinder.

In the electroporation method, a cell dispersion solution is mixed with a solution containing a site-specific nuclease or the like, and a voltage pulse is applied to this mixed solution. This makes it possible to perforate a cell wall and introduce a site-specific nuclease or the like into the cell. In this case, the mutagenesis apparatus may include a holder that holds the mixed solution containing cells, the site-specific nuclease, or the like, and an electrode that applies a voltage pulse to the mixed solution.

Other methods may be used to introduce the site-specific nuclease or the like into the cells. For example, a chemical scheme can be used to introduce the site-specific nuclease or the like into the cells. For example, the site-specific nuclease or the like may be introduced into protoplasts whose cell walls have been processed with cell wall-degrading enzymes. Alternatively, cells may be infected with a virus containing a vector modified to produce the site-specific nuclease or the like, such as a vector modified to produce Cas9 and sgRNA, so that the site-specific nuclease or the like is produced in the cell.

The genome-editing apparatus 11A further includes an analysis apparatus. The analysis apparatus analyzes what kind of mutation has occurred in the genomic DNA in the mutagenesis apparatus. For example, the analysis apparatus analyzes a sequence of genomic DNA into which mutation has been introduced in the mutagenesis apparatus.

The editing result-recording apparatus 12A records results of the genome editing performed by the genome-editing apparatus 11A. The editing result-recording apparatus 12A, for example, records information including analysis results of the analysis apparatus. According to one example, this information includes information on the cleaving position, mutation occurring at the cleaving position, presence or absence of mutations occurring at a position other than the cleaving position, and mutation occurring at the position other than the cleaving position when the mutation occurs at the position. According to another example, this information records a sequence of genomic DNA into which mutation has been introduced. The editing result-recording apparatus 12A can include, for example, a non-volatile storage apparatus such as a hard disk drive and a solid state drive.

The seedling-growing apparatus 20 obtains seedling from the mutant generated in the genome-editing apparatus 11A. The seedling-growing apparatus 20 can be omitted.

The growing system 40 includes one or more growing apparatuses 41 and an integrated control apparatus 42. The number n of growing apparatuses 41 is preferably two or more. The number n does not have an upper limit. Here, as an example, it is assumed that the growing system 40 includes a plurality of growing apparatuses 41.

Each of the growing apparatuses 41 includes a growth environment chamber 410, an environment control apparatus 420, and a growth situation-monitoring apparatus 430.

Each of the environment chambers 410 grows one or more of the plurality of living bodies 30 respectively derived from the plurality of mutants. As described above, the seedling-growing apparatus 20 can be omitted. Therefore, the living body 30 may be the seedling obtained in the seedling-growing apparatus 20, or may a mutant generated in the genome-editing apparatus 11A.

Each environment control apparatus 420 controls an environment in the environment chamber 410. According to one example, the environment control apparatus 420 controls one or more of a luminous intensity of a light source illuminating the living body 30 inside the environment chamber 410, a temperature of a gas phase and/or liquid phase inside the environment chamber 410, a humidity inside the environment chamber 410, a liquid phase acidity and electrical conductivity inside the environment chamber 410, and a water feed rate. The environment control apparatus 420 can include one or more of environment adjustment apparatuses such as a light source, a heater, a cooler, a humidifier, a dehumidifier, a pH adjuster, and a water supply apparatus. The environment control apparatus 420 may further include one or more of environment-monitoring apparatuses such as a light sensor, a temperature sensor, a humidity sensor, a pH meter, a conductivity meter, and a flow meter.

Each growth situation-monitoring apparatus 430 monitors the growth situation of the living body 30 in the environment chamber 410. According to one example, the growth situation-monitoring apparatus 430 estimates a degree of growth of the living body 30 through image analysis, measures one or more of mass, photosynthetic rate, transpiration rate, respiration rate, CO₂ application rate, and water absorption rate, or perform a combination thereof to perform monitoring. The growth situation-monitoring apparatus 430 can include one or more of an image sensor, a mass meter, a photosynthetic rate measurement apparatus, a transpiration meter, a respirometer, and a flow meter.

Each growing apparatus 41 further includes a recording apparatus (not illustrated). The recording apparatus records at least one of operation information of the environment adjustment apparatus and environment information acquired by the environment-monitoring apparatus, and the growth situation information acquired by the growth situation-monitoring apparatus 430. The storage apparatus can include, for example, a non-volatile storage apparatus such as a hard disk drive and a solid state drive.

The integrated control apparatus 42 individually controls an operation of the environment control apparatus 420. The integrated control apparatus 42 makes operations of the environment control apparatuses 420 different between the growing apparatuses 41, thereby making it possible to make the growth environments of the living body 30 different between the growing apparatuses 41.

The integrated control apparatus 42 includes a processing unit, a main storage apparatus, an auxiliary memory apparatus, an input apparatus, and an output apparatus.

The processing unit includes a central processing unit. The processing unit reads a program, receives a command and information transmitted from the input apparatus, and the processing unit performs calculation processing according to the program to control an operation of the environment control apparatus 420.

The main storage apparatus temporarily stores information to be processed, programs, calculation results, and the like. The main storage apparatus includes a volatile memory such as a random access memory.

The auxiliary storage apparatus is a non-volatile storage apparatus. The auxiliary storage apparatus can store programs and various pieces of data for a long period of time. The auxiliary storage apparatus includes, for example, one or both of a hard disk drive and a solid state drive.

The input apparatus allows the operator to input growing conditions to be set for each of the growing apparatus 41. The input apparatus includes, for example, one or more of a keyboard, a mouse, and a touch panel.

The output apparatus enables the operator to recognize the growing conditions or the like input by the operator. The output apparatus may allow the operator to further recognize one or more of the operation information of the environment adjustment apparatus, the environment information acquired by the environment-monitoring apparatus, and the growth situation information acquired by the growth situation-monitoring apparatus 430. The output apparatus is, for example, a display apparatus such as a liquid crystal display apparatus and an organic electroluminescence (EL) display apparatus.

The integrated control apparatus 42 can be omitted. In this case, each growing apparatus 41 is provided with a control apparatus that controls the operation of the environment control apparatus 420.

As will be described below, the processing apparatus 50, for example, obtains a predictive model that predicts the growth situation of a living body from a genome mutation and a growth environment, from genome mutation data, growth environment data, and growth situation data using machine learning such as deep learning. The processing apparatus 50 includes a data acquisition unit 51, a correlation analysis unit 52, a predictive model-generating unit 53, and a display and recording unit 54. These will be described in detail below.

The processing apparatus 50 can include, for example, a network apparatus, a processing unit, a main storage apparatus, an auxiliary storage apparatus, and a display apparatus. The network apparatus allows the processing apparatus 50 to be wired or wirelessly connected to the editing result-recording apparatus 12A of the genome-editing unit IGA and the recording apparatus of the growing apparatus 41. The processing unit includes a central processing unit. The processing unit reads a program, receives a command and information transmitted from the input apparatus, and the processing unit performs calculation processing according to the program. The main storage apparatus temporarily stores information to be processed, programs, calculation results, and the like. The main storage apparatus includes a volatile memory such as a random access memory. The auxiliary storage apparatus is a non-volatile storage apparatus. The auxiliary storage apparatus can store programs and various pieces of data for a long period of time. The auxiliary storage apparatus includes, for example, one or both of a hard disk drive and a solid state drive. The display apparatus is, for example, a display apparatus such as a liquid crystal display apparatus or an organic EL display apparatus.

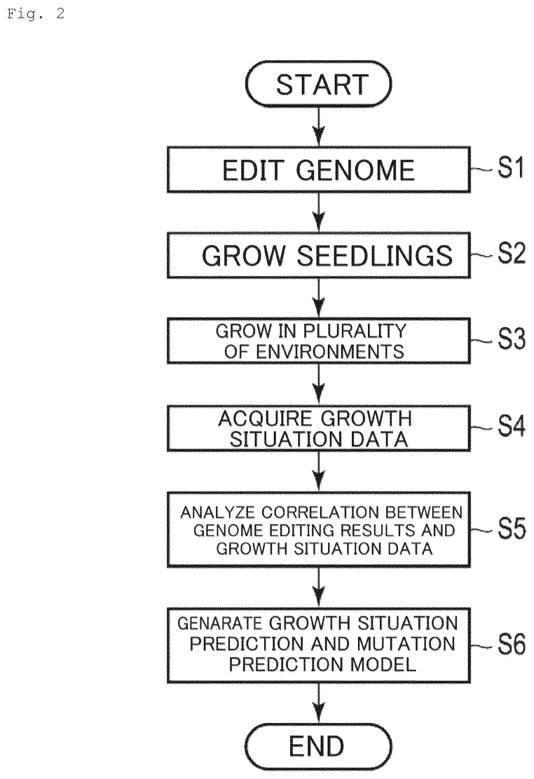

FIG. 2 is a flowchart of a predictive model-generating method according to the first embodiment of the present invention. Hereinafter, a method of generating a predictive model using the predictive model-generating system 1A described with reference to FIG. 1 will be described as an example of the method of FIG. 2.

First, the genome-editing apparatus 11A generates a plurality of mutants with different genetic information through genome editing of the living body (here, a plant) (step S1). The genome-editing apparatus 11A may generate these mutants simultaneously or sequentially. Here, as an example, the genome-editing apparatus 11A sequentially generates mutants.

The genome-editing apparatus 11A analyzes what kind of mutation has occurred in the genomic DNA. The editing result-recording apparatus 12A records results of the genome editing performed by the genome-editing apparatus 11A.

Next, the seedling-growing apparatus 20 grows the seedliings from each mutant (step S2). The seedling-growing apparatus 20 may simultaneously grow seedlings derived in the plurality of mutants, from a plurality of mutants. Alternatively, the seedling-growing apparatus 20 may sequentially grow the seedlings derived in the plurality of mutants, from the plurality of mutants. Here, as an example, it is assumed that the seedling-growing apparatus 20 sequentially grows the seedlings derived in the plurality of mutants, from the plurality of mutants. Step S2 can be omitted.

Next, the growing system 40 grows a plurality of living bodies 30 respectively derived from the plurality of mutants (here respective seedlings) under different environments (step S3). For example, the growing system 40 grows the living body 30 from the beginning to the end of the growth. Alternatively, the growing system 40 grows the living body 30 so that a life cycle thereof is repeated.

The growing system 40 may simultaneously grow a plurality of living bodies 30 respectively derived from the plurality of mutants under different environments. Alternatively, the growing system 40 may grow living bodies 30 derived from a certain mutant under different environments, and then, grow living bodies 30 derived from another mutant under different environments. Here, as an example, it is assumed that the growing system 40 grows living bodies 30 derived from a certain mutant under different environments, and then grows living body 30 derived from another mutant under different environments.

Each growth situation-monitoring apparatus 430 monitors the growth situation of the living bodies 30 in the environment chamber 410. The recording apparatus included in each growing apparatus 41 records at least one of the operation information of the environment adjustment apparatus and the environment information acquired by the environment-monitoring apparatus, and the growth situation information acquired by the growth situation-monitoring apparatus 430.

The data acquisition unit 51 acquires the genome mutation data from the genome-editing apparatus 11A via the editing result-recording apparatus 12A. The data acquisition unit 51 acquires at least one of the operation information and the environment information of the environment adjustment apparatus and the growth situation information, that is, the growth environment data and the growth situation data from the recording apparatus included in the growing apparatus 41 (step S4).

The correlation analysis unit 52 statistically analyzes a correlation among the genome mutation data, the growth environment data, and the growth situation data (step S5). This analysis is performed after the genome mutation data, the growth environment data, and the growth situation data are obtained for the plurality of living bodies 30 respectively derived from the plurality of mutants.

The correlation analysis unit 52 sets one or more variables included in the growth situation data as objective variables, and selects the feature quantity to be used as the objective variables from a feature quantity included in the genome mutation data and the growth environment data. For example, the correlation analysis unit 52 selects the feature quantity having the highest correlation with the growth of the living body 30 from among the feature quantities included in the genome mutation data and the growth environment data. Alternatively, the correlation analysis unit 52 selects a combination having the highest correlation with the growth of the living body 30 from combinations of these feature quantities. The correlation analysis unit 52 generates teacher data including the explanatory variables and objective variables selected in this way. The selection of the feature quantities may be performed by the operator on the basis of analysis result of the correlation analysis unit 52.

The predictive model-generating unit 53 causes a machine-learning model that predicts the growth situation of the living body from the genome mutation and the growth environment to perform learning using teacher data. Accordingly, the predictive model-generating unit 53 obtains the machine-learning model that has performed the learning as a predictive model (step S6). Here, the growth situation to be predicted includes, for example, a growth rate of at least some organs, a trait thereof, or both.

The display and recording unit 54 records information on the predictive model obtained as described above and displays at least part of the information. The display and recording unit 54 can further display a result of the prediction using the predictive model. For example, when the growth situation of the living body 30 can be predicted from the genome mutation data and the virtual growth environment data before the processing apparatus 50 starts growing the living body 30, the display and recording unit 54 can display the prediction result.

Using the predictive model obtained in this way, for example, it is possible to predict the growth situation of the living body according to the growth environment for a novel mutant. Therefore, it becomes easy to determine the growth environment optimal for the growth of the living body. For example, it is also possible to automatically determine the growth environment optimal for the growth of the living body using this predictive model. Therefore, it is possible to efficiently grow the living body.

Alternatively, using this predictive model, it is also possible to estimate what kind of genome editing should be performed in order to realize a desired growth situation for each growth environment. This makes efficient breed improvement possible.

For example, first, the predictive model is used to predict the growth situation of the living body according to the growth environment for a plurality of virtual mutants. Next, from among these virtual mutants, those predicted to achieve a desirable growth situation under any growth environment are selected. Genome editing that can produce the selected mutant is actually performed, and the living body derived from the resulting mutant is grown under the growth environment, and it is confirmed whether an expected growth situation is achieved. For example, efficient breed improvement becomes possible by performing prediction and verification in such a procedure.

Further, in a generalized linear model, a relationship between genome mutation caused by genome editing and the growth situation cannot be completely represented. In creating the predictive model, statistical analysis is performed on the correlation among the genome mutation data, the growth environment data, and the growth situation data, and, for example, two or more feature quantities are selected as explanatory variables. Therefore, prediction with high accuracy is possible.

Here, the machine-learning model predicts the growth situation of the living body from the genome mutation and the growth environment. For example, when the living body 30 has been grown so that a life cycle of the living body 30 is repeated, and mutation has occurred in the living body 30, it is possible to confirm the occurrence of the mutation from the growth situation. Therefore, the machine-learning model may predict a mutagenesis situation of the living body from the genome mutation and the growth environment.

### <2> Second Embodiment

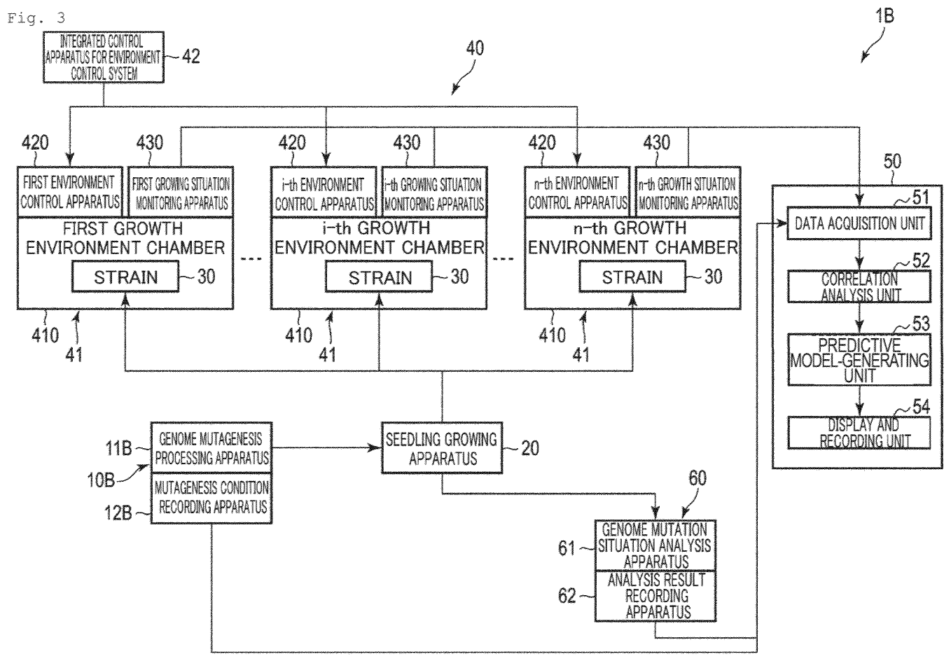

FIG. 3 is a block diagram of a predictive model-generating system according to a second embodiment of the present invention.

A predictive model-generating system 1B illustrated in FIG. 3 is the same as the predictive model-generating system 1A described above, except that the predictive model-generating system 1B includes a genome mutagenesis-processing unit 10B and a genome mutation situation analysis unit 60 instead of the genome-editing unit 10A.

The genome mutagenesis-processing unit 10B includes a genome mutagenesis-processing apparatus 11B and a mutagenesis condition-recording apparatus 12B.

The genome mutagenesis-processing apparatus 11B generates a plurality of processing bodies by performing genome mutagenesis-processing on a processed body. The processed body is a living body or a part thereof. Here, as an example, it is assumed that the processed body is a plant or a part thereof. According to one example, the processed body is a plant body, a tissue such as a shoot apical meristem, or a callus. The genome mutagenesis-processing apparatus 11B induces genome mutation in the processed body, for example, by irradiating the processed body with ultraviolet light or radiation. In this case, the genome mutagenesis-processing apparatus 11B includes an ultraviolet or radiation source.

The mutagenesis condition-recording apparatus 12B records conditions for genome mutagenesis processing in the genome mutagenesis-processing apparatus 11B. For example, the mutagenesis condition-recording apparatus 12B records one or more of a type of radiation source, wavelength, energy, irradiation time, and an irradiation dose. The mutagenesis condition-recording apparatus 12B can include, for example, a non-volatile storage apparatus such as a hard disk drive and a solid state drive.

The genome mutation situation analysis unit 60 includes a genome mutation situation analysis apparatus 61 and an analysis result-recording apparatus 62.

The genome mutation situation analysis apparatus 61 analyzes the genome mutation situation of each processing body. That is, the genome mutation situation analysis apparatus 61 analyzes whether or not a genome mutation has occurred for each of the processing bodies. The genome mutation situation analysis apparatus 61 analyzes what kind of mutation has occurred in the genomic DNA for at least a processing body with genome mutation.

The analysis result-recording apparatus 62 records results of the analysis of the genome mutation situation analysis apparatus 61. The analysis result-recording apparatus 62 can include, for example, a non-volatile storage apparatus such as a hard disk drive and a solid state drive.

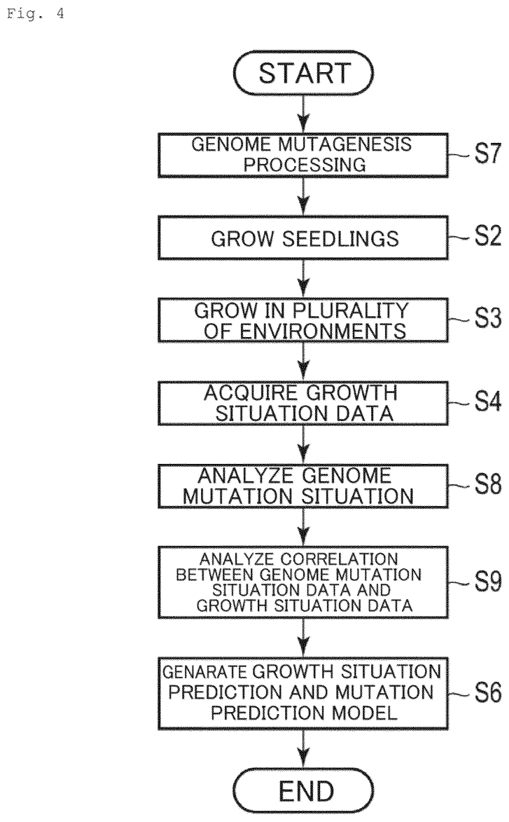

FIG. 4 is a flowchart of a predictive model-generating method according to the second embodiment of the present invention. Hereinafter, a method of generating a predictive model using the predictive model-generating system 1B described with reference to FIG. 3 will be described as an example of the method of FIG. 4.

First, the genome mutagenesis-processing apparatus 11B generates a plurality of processing bodies through genome mutagenesis processing of a processed body (here, a plant or a part thereof) (Step S7). The mutagenesis condition-recording apparatus 12B records conditions for genome mutagenesis processing in the genome mutagenesis-processing apparatus 11B.

Next, the seedling-growing apparatus 20 and the growing system 40 sequentially perform steps S2 to S4, as in the method described with reference to FIG. 2, except that the processed body is used instead of the mutant.

The genome mutation situation analysis apparatus 61 analyzes the genome mutation situation for each processing body (step S8). The analysis result-recording apparatus 62 records results of the analysis of the genome mutation situation analysis apparatus 61. The data acquisition unit 51 acquires the genome mutation data from the genome mutation situation analysis apparatus 61 via the analysis result-recording apparatus 62.

The correlation analysis unit 52 statistically analyzes the correlation among the genome mutation data, the growth environment data, and the growth situation data (Step S9). Step S9 is the same as step S5 described with reference to FIG. 1 and FIG. 2 except that the data acquisition unit 51 acquires the genome mutation data from the analysis result-recording apparatus 62 instead of acquiring the genome mutation data from the editing result-recording apparatus 12A. The predictive model-generating unit 53 performs step S6 described with reference to FIG. 1 and FIG. 2. The display and recording unit 54 records the information on the predictive model obtained as described above and displays at least part of the information. The display and recording unit 54 can further display a result of the prediction using the predictive model. For example, when the growth situation of the living body 30 can be predicted from the genome mutation data and the virtual growth environment data before the processing apparatus 50 starts growing the living body 30, the display and recording unit 54 can display the prediction result.

Using the predictive model obtained in this way, for example, it is possible to predict the growth situation of the living body according to the growth environment for a novel mutant. Therefore, it becomes easy to determine the growth environment optimal for the growth of the living body. For example, it is also possible to automatically determine the growth environment optimal for the growth of the living body using this predictive model. Therefore, it is possible to efficiently grow the living body.

Alternatively, using this predictive model, it is also possible to estimate what kind of genome mutation should be generated in order to realize a desired growth situation for each growth environment. This makes efficient breed improvement possible.

The processing apparatus 50 may further create a predictive model that predicts genome mutation from genome mutagenesis-processing conditions, from genome mutagenesis-processing data and the genome mutation data. Here, the genome mutagenesis-processing data is acquired from the genome mutagenesis-processing apparatus 11B via the mutagenesis condition-recording apparatus 12B. Use of this predictive model enables more efficient breed improvement.

Here, the machine-learning model predicts the growth situation of the living body from the genome mutation and the growth environment. For example, when the living body 30 has been grown so that a life cycle of the living body 30 is repeated, and mutation has occurred in the living body 30, it is possible to confirm the occurrence of the mutation from the growth situation. Therefore, the machine-learning model may predict the mutagenesis situation of the living body from the genome mutation and the growth environment.

### <3> Third Embodiment

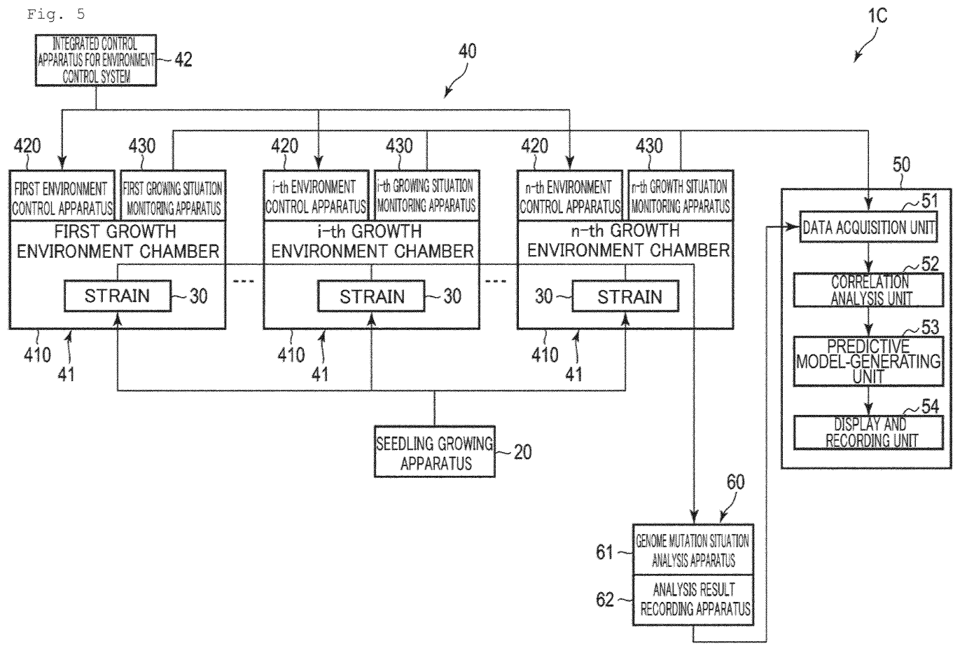

FIG. 5 is a block diagram of a predictive model-generating system according to a third embodiment of the present invention.

A predictive model-generating system 1C illustrated in FIG. 5 is the same as the predictive model-generating system 1B described above except that the genome mutagenesis-processing unit 108 is omitted, and the genome mutation situation analysis unit 60 analyzes a genome mutation situation of the living body 30 grown in the growing system 40.

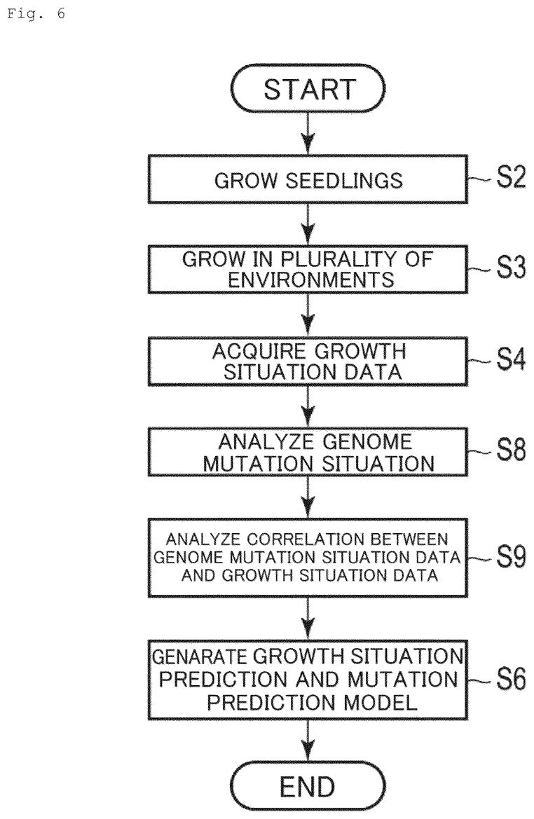

FIG. 6 is a flowchart of a predictive model-generating method according to the third embodiment of the present invention. Hereinafter, a method of generating a predictive model using the predictive model-generating system 1C described with reference to FIG. 5 will be described as an example of the method of FIG. 6.

First, step S2 described above is performed as in the method described with reference to FIG. 2 except that the seedling-growing apparatus 20 grows seedlings derived from a wild-type strain or a single mutant, instead of growing seedlings derived from a plurality of mutants.

The growing system 40 sequentially performs steps S3 and S4, as in the method described with reference to FIG. 2. Preferably, the growing system 40 grows the living body 30 such that a life cycle of the living body 30 is repeated in each growing apparatus 41.

The genome mutation situation analysis apparatus 61 performs step S8, as in the method described with reference to FIG. 4. The analysis result-recording apparatus 62 records results of the analysis of the genome mutation situation analysis apparatus 61. The data acquisition unit 51 acquires genome mutation situation data from the genome mutation situation analysis apparatus 61 via the analysis result-recording apparatus 62.

The correlation analysis unit 52 performs step S9 similar to step S5 described with reference to FIG. 1 and FIG. 2 except that the correlation analysis unit 52 statistically analyzes the correlation between the genome mutation situation data, the growth environment data, and the growth situation data, instead of statistically analyzing the correlation between the genome-editing data, the growth environment data, and the growth situation data.

The predictive model-generating unit 53 performs step S6 described with reference to FIG. 1 and FIG. 2. The display and recording unit 54 records the information on the predictive model obtained as described above and displays at least part of the information.

Using the predictive model obtained in this way, for example, it is possible to predict the growth situation of the living body according to the growth environment for a novel mutant. Therefore, it becomes easy to determine the growth environment optimal for the growth of the living body. For example, it is also possible to automatically determine the growth environment optimal for the growth of the living body using this predictive model. Therefore, it is possible to efficiently grow the living body.

Alternatively, using this predictive model, it is also possible to estimate what kind of genome mutation should be generated in order to realize a desired growth situation for each growth environment. This makes efficient breed improvement possible.

The processing apparatus 50 may further create a predictive model for predicting the mutagenesis situation from the growth environment, from the growth environment data and the genome mutation state data.

### <4> Modification Example

Various modifications are possible for the first to third embodiments.

For example, the machine-learning model predicts the growth situation of the living body from the genome mutation and the growth environment. The machine-learning model may be a model that predicts what kind of genome mutation has occurred in the living body from the growth environment and the living body growth situation.

The genome mutagenesis-processing apparatus 11B may be omitted from the predictive model-generating system 1B, and a library of mutants generated using a transposon may be used instead of generating a plurality of processing bodies by genome mutagenesis processing.

The living body to be grown may be a living body other than a land plant. For example, a water tank may be used as the environment chamber 410, and algae, fish, shellfish, or the like may be grown as the living body 30. In this case, the environment control apparatus 420 is provided with a water temperature sensor.

The living body to be grown may be an animal or fungus. In this case, a juvenile-growing apparatus is installed instead of the seedling-growing apparatus 20.

Thus, the living body to be grown may be any creature having a genome, such as a terrestrial plant, an aquatic plant, a terrestrial animal, and an aquatic animal.

The present invention is not limited to the embodiments described above, and it is obvious that many modifications and combinations can be made by those skilled in the art within the technical spirit of the present invention. Further, each apparatus may also have a communication function, a recording function, a display function, a control function, and a calculation function, although they are not individually shown.

### REFERENCE SIGNS LIST

1A Predictive model-generating system
1B Predictive model-generating system
1C Predictive model-generating system
10A Genome-Editing unit
10B Genome mutagenesis-processing unit
11A Genome-editing apparatus
11B Genome mutagenesis-processing apparatus
12A Editing result-recording apparatus
12B Mutagenesis condition-recording apparatus
20 Seedling-growing apparatus
30 Living body
40 Growing system
41 Growing apparatus
42 Integrated control apparatus
50 Processing apparatus
51 Data acquisition unit
52 Correlation analysis unit
53 Predictive model-generating unit
54 Display and recording unit
60 Genome mutation situation analysis unit
61 Genome mutation situation analysis apparatus
62 Analytical result-recording apparatus
410 Environment chamber
420 Environment control apparatus
430 Growth situation-monitoring apparatus

1. A predictive model-generating system comprising:
a growing system including one or more growing apparatuses, each of the one or more growing apparatuses including an environment chamber configured to grow one or more of a plurality of living bodies respectively derived from a plurality of mutants, an environment control apparatus configured to control an environment in the environment chamber, and a growth situation-monitoring apparatus configured to monitor a growth situation of the living body in the environment chamber, and the one or more growing apparatuses growing each of the plurality of living bodies under different environments; and
a processing apparatus configured to acquire growth environment data and growth situation data in each of the one or more growing apparatuses from the growing system, generate teacher data from the genome mutation data of the plurality of mutants, the growth environment data, and the growth situation data, cause a machine-learning model for predicting the growth situation or mutagenesis situation of the living body from the genome mutation and the growth environment or predicting the genome mutation from the growth environment and the growth situation of the living body, to perform learning with the teacher data, and obtain the machine-learning model that has completed the learning as a predictive model.

2. The predictive model-generating system according to claim 1, further comprising:
a genome-editing apparatus configured to generate the plurality of mutants through genome editing.

3. The predictive model-generating system according to claim 1, further comprising:
a genome mutagenesis-processing apparatus configured to generate a plurality of processing bodies containing the plurality of mutants through genome mutagenesis processing; and
a genome mutation situation analysis apparatus configured to analyze the genome mutation situation of each of the plurality of processing bodies.

4. A predictive model-generating system comprising:
a growing system including one or more growing apparatuses, each of the one or more growing apparatuses including an environment chamber configured to grow living bodies, an environment control apparatus configured to control an environment in the environment chamber, and a growth situation-monitoring apparatus configured to monitor a growing condition of the living body in the environment chamber, and the one or more growing systems includes a growing system configured to grow the living body under different environments;
a genome mutation situation analysis apparatus configured to analyze a genome mutation situation for the living bodies grown in the one or more growing apparatuses; and
a processing apparatus configured to acquire genome mutation situation data from the genome mutation situation analysis apparatus, acquire growth environment data and growth situation data in each of the one or more growing apparatuses from the growing system, generate teacher data from the genome mutation data, the growth environment data, and the growth situation data, cause a machine-learning model for predicting the growth situation or mutagenesis situation of the living body from the genome mutation and the growth environment or predicting the genome mutation from the growth environment and the growth situation of the living body, to perform learning with the teacher data, and obtain the machine learning-model that has completed the learning as a predictive model.

5. A method of generating a predictive model comprising:
growing a plurality of living bodies respectively derived from a plurality of mutants under different environments; and
acquiring genome mutation data for the plurality of mutants, acquiring growth environment data and growth situation data of the plurality of living bodies, generating teacher data from the genome mutation data, the growth environment data, and the growth situation data, causing a machine-learning model for predicting the growth situation or mutagenesis situation of the living body from the genome mutation and the growth environment or predicting the genome mutation from the growth environment and the growth situation of the living body, to perform learning with the teacher data, and obtaining the machine-learning model that has completed the learning as a predictive model.

6. (canceled)

7. A prediction method comprising:
generating the predictive model by the method according to claim 5; and
predicting the growth situation or mutagenesis situation of the living body or genome mutation using the predictive model.
